(19) United States
(12) Patent Application Publication
(10) Pub. No.: US 2014/0118691 A1
(43) Pub. Date: May 1, 2014

(54) OPHTHALMIC APPARATUS, IMAGING CONTROL APPARATUS, AND IMAGING CONTROL METHOD

(71) Applicant: CANON KABUSHIKI KAISHA, Tokyo (JP)

(72) Inventors: Manabu Wada, Kawasaki-shi (JP); Hajime Nakajima, Tokyo (JP)

(73) Assignee: CANON KABUSHIKI KAISHA, Tokyo (JP)

(21) Appl. No.: 14/061,920

(22) Filed: Oct. 24, 2013

(30) Foreign Application Priority Data
Nov. 1, 2012 (JP) 2012-242213

(51) Int. Cl. A61B 3/12; H04N 5/232

(52) U.S. Cl. CPC A61B 3/12; H04N 5/23212
USPC 351/206; 348/78

(57) ABSTRACT

An imaging control apparatus performs focusing operation to set an imaging optical system for an image sensor in an in-focus state with respect to an object illuminated by a light source by using an object image obtained by imaging the object using the image sensor. The imaging control apparatus changes an imaging setting so as to set the signal-to-noise ratio of an acquired object image during execution of focusing operation for this focusing higher than that during non-execution of the focusing operation.

### BACKGROUND OF THE INVENTION

1. Field of the Invention

The present invention relates to an ophthalmic apparatus, an imaging control apparatus, and an imaging control method.

2. Description of the Related Art

In general, when using an ophthalmic apparatus typified by a non-mydriatic fundus camera, the operator performs positioning and focusing between the apparatus and the eye to be examined in the upward, downward, leftward, rightward, forward, and backward directions while seeing the fundus observation image captured by an image sensor. Recently, an ophthalmic apparatus having an autofocus function is widely known, which is designed to automatically perform focusing by using the fundus observation image captured by an image sensor.

The autofocus schemes for ophthalmic apparatuses can be roughly classified into two types. One is a scheme of performing autofocus by projecting split indices on the pupil of the eye to be examined and detecting a positional relationship between captured index images by image processing as disclosed in Japanese Patent Laid-Open No. 5-95907. In this case, the autofocus scheme using index images is defined as an index image autofocus scheme. This index image autofocus scheme can perform accurate focusing in the splitting direction of indices on the pupil with respect to the ametropia such as astigmatism of the optical system of the eye to be examined, but cannot perform accurate focusing in directions other than the splitting direction of the indices on the pupil.

The other one is a scheme of performing autofocus by detecting tone differences on a fundus observation image itself by image processing without using any index images projected on the fundus of the eye to be examined when performing autofocus, as disclosed in Japanese Patent Laid-Open No. 2011-50532. In this case, the autofocus scheme using a fundus image is defined as a fundus image autofocus scheme. This fundus image autofocus scheme can minimize an error of the ametropia such as astigmatism of the optical system of the eye to be examined described with reference to the index image autofocus scheme.

However, the fundus image autofocus scheme using the fundus observation image captured by an image sensor is susceptible to the influence of noise of the image sensor because the tone differences on a fundus observation image as a focusing target are small. This leads to a deterioration in focusing accuracy. It is possible to improve the focusing accuracy by increasing the signal-to-noise ratio (S/N ratio) between a fundus observation image and noise of the image sensor. For example, such a signal-to-noise ratio can be increased by increasing the illumination light amount of an observation light source. It is not, however, always necessary to increase the signal-to-noise ratio (S/N ratio) between a fundus observation image and noise of an image sensor during observation, for example, when positioning the eye to be examined with respect to the apparatus in the upward, downward, leftward, rightward, forward, and backward directions. If, therefore, the illumination light amount of the observation light source is kept large during observation, an unnecessarily heavy burden is imposed on the object.

### SUMMARY OF THE INVENTION

An embodiment of the present specification implements accurate focusing by using an object image without imposing any unnecessarily heavy burden on an object in an ophthalmic apparatus or the like.

According to one aspect of the present invention, there is provided an imaging control apparatus comprising: a focusing unit configured to perform focusing operation to set an imaging optical system in an in-focus state with respect to an object illuminated by a light source by using an object image obtained by imaging the object using an image sensor; and a change unit configured to change an imaging setting so as to set a signal-to-noise ratio of the object image acquired by the imaging during execution of focusing operation by the focusing unit higher than that during non-execution of the focusing operation.

Also, according to another aspect of the present invention, there is provided an ophthalmic apparatus comprising:

the above-described imaging control apparatus;

the light source;

the image sensor; and

the imaging optical system,

wherein a fundus image is captured as the object image.

Furthermore, according to another aspect of the present invention, there is provided an imaging control method comprising: a step of causing focusing means to perform focusing operation to set an imaging optical system in an in-focus state with respect to an object illuminated by a light source by using an object image obtained by imaging the object using an image sensor; and a step of causing change means to change an imaging setting so as to set a signal-to-noise ratio of the object image acquired by the imaging during execution of the focusing operation higher than that during non-execution of the focusing operation.

Further features of the present invention will become apparent from the following description of exemplary embodiments with reference to the attached drawings.

### BRIEF DESCRIPTION OF THE DRAWINGS

FIG. 1 is a block diagram showing an example of the arrangement of a non-mydriatic fundus camera according to the first embodiment;

FIG. 2 is a flowchart showing the operation of the non-mydriatic fundus camera according to the first embodiment;

FIG. 3 is a view showing a fundus observation image and a focusing evaluation area for fundus image autofocus;

FIG. 4 is a view showing a fundus observation image in a focusing evaluation area;

FIG. 5 is a graph showing the tone values of a fundus observation image without any influence of noise of an image sensor;

FIG. 6 is a graph showing the tone values of a fundus observation image when noise of the image sensor is superimposed;

FIG. 7 is a block diagram showing an example of the arrangement of a non-mydriatic fundus camera according to the second embodiment;

FIG. 8 is a flowchart showing the operation of the non-mydriatic fundus camera according to the second embodiment;

FIG. 9 is a view showing a fundus observation image, index images, and a focusing evaluation area 23;

FIG. 10 is a view showing index images in a focusing evaluation area; and

FIG. 11 is a graph showing the tone values of an index image 22.

### DESCRIPTION OF THE EMBODIMENTS

Several preferred embodiments of the present invention will be described below with reference to the accompanying drawings. Each embodiment of the present invention will be described below by exemplifying a case in which an imaging control apparatus according to the present invention is applied to an ophthalmic apparatus, especially a non-mydriatic fundus camera.

### First Embodiment

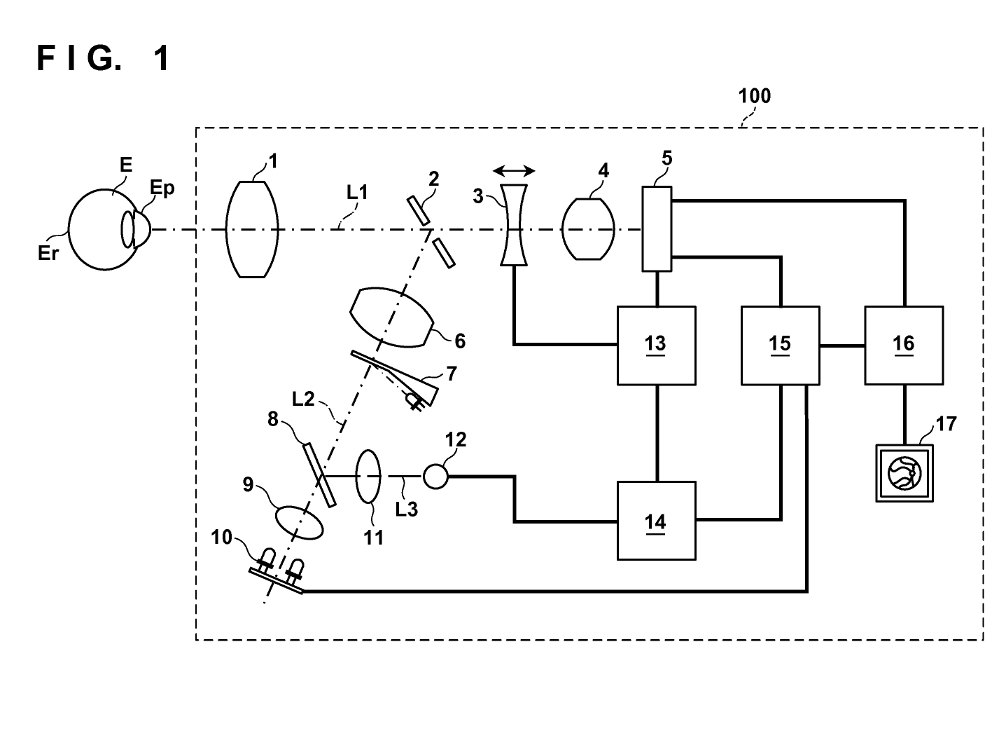

FIG. 1 is a block diagram showing an example of the arrangement of a non-mydriatic fundus camera 100 according to the first embodiment. The non-mydriatic fundus camera 100 according to this embodiment has a function of performing fundus image autofocus. The arrangement of the non-mydriatic fundus camera 100 will be described first with reference to FIG. 1.

An objective lens 1, a perforated mirror 2, a focus lens 3, an imaging lens 4, and an image sensor 5 are sequentially arranged on an optical axis L1 extending to a fundus Er of an eye E to be examined and constitute an imaging optical system. This embodiment exemplifies the imaging optical system forming a non-mydriatic fundus camera. Such an imaging optical system forms a fundus imaging optical system for the eye E. On the other hand, a lens 6, an index projection part 7, a dichroic mirror 8, a condenser lens 9, and an observation light source 10 are arranged on an optical axis L2 in the reflecting direction of the perforated mirror 2. In addition, a condenser lens 11 and an imaging light source 12 are arranged on an optical axis L3 in the reflecting direction of the dichroic mirror 8. The arrangements on the optical axes L2 and L3 constitute an illumination optical system. Such an illumination optical system is an example of a fundus illumination optical system forming the non-mydriatic fundus camera 100.

The dichroic mirror 8 has the property of transmitting light in the wavelength range of the observation light source 10 and reflecting light in the wavelength range of the imaging light source 12. The observation light source 10 is a light source in which a plurality of LEDs are arranged and which irradiates the eye to be examined with light having a wavelength in the infrared region. The imaging light source 12 is a light source which irradiates the fundus Er with light having a wavelength in the visible region.

The non-mydriatic fundus camera 100 further includes a fundus image autofocus part 13, a fundus camera control part 14, an SN control part 15, a display image processing part 16, and a display part 17. More specifically, the fundus image autofocus part 13 is connected to the focus lens 3, the image sensor 5, and the fundus camera control part 14. The fundus image autofocus part 13 calculates a focusing evaluation value from an image from the image sensor 5 and drives the focus lens 3 based on instructions from the fundus camera control part 14. The SN control part 15 is connected to the image sensor 5, the observation light source 10, the fundus camera control part 14, and the display image processing part 16, and sets the amplification factor of the image sensor 5 and the emitted light amount of the observation light source 10 based on instructions from the fundus camera control part 14. The fundus camera control part 14 is connected to the imaging light source 12, the fundus image autofocus part 13, and the SN control part 15, and performs overall control such as light emission control on the imaging light source 12 and operation start/end control on the fundus image autofocus part 13 and the SN control part 15. The display image processing part 16 is connected to the image sensor 5 and the display part 17, and performs image processing for an image from the image sensor 5 to display the image on the display part 17. The fundus image autofocus part 13, the fundus camera control part 14, the SN control part 15, and the display image processing part 16 described above constitute the imaging control part of the non-mydriatic fundus camera 100.

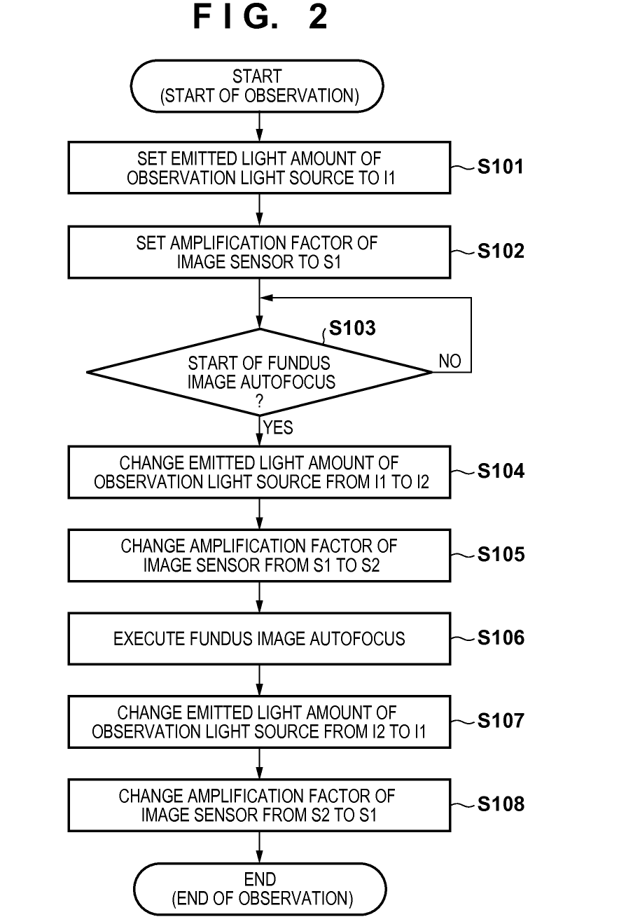

Operation from observation to imaging in the non-mydriatic fundus camera 100 having the above arrangement according to this embodiment will be described below. Observing operation will be described first with reference to the flowchart shown in FIG. 2. The flowchart of FIG. 2 shows the operation of fundus image autofocus part 13, fundus camera control part 14, and SN control part 15.

When the operator positions the eye E in front of the objective lens 1 and the apparatus starts observing operation in accordance with predetermined operation by the operator, the SN control part 15 sets first the emitted light amount of the observation light source 10 to I1 (step S101). When the observation light source 10 emits light at the emitted light amount I1 set by the SN control part 15, the observation illumination light passes through the fundus illumination optical system extending from the observation light source 10 to the objective lens 1 and illuminates the fundus Er via a pupil Ep of the eye E. The reflected light from the fundus Er illuminated by the observation light source 10 passes through the fundus imaging optical system extending to the objective lens 1, the perforated mirror 2, the focus lens 3, and the imaging lens 4 and reaches the image sensor 5.

At the same time as the setting of the observation light source 10, the SN control part 15 sets the amplification factor of the image sensor 5 to S1 (step S102). The image sensor 5 captures a fundus observation image with the set amplification factor S1. The display image processing part 16 applies processing such as monochromatization processing or gamma curve calculation to the fundus observation image and displays the resultant image on the display part 17. The operator moves the non-mydriatic fundus camera 100 upward, downward, leftward, rightward, forward, and backward by operating a console (not shown) while seeing the fundus observation image displayed on the display part 17, thereby performing positioning between the eye E and the non-mydriatic fundus camera 100 in the upward, downward, leftward, rightward, forward, and backward directions.

Upon determining the completion of positioning when, for example, the positional relationship between the eye E and the non-mydriatic fundus camera 100 satisfies a predetermined relationship, the apparatus issues an instruction to start fundus image autofocus. That is, the apparatus automatically starts fundus image autofocus in response to the completion of positioning (YES in step S103). Note that the present invention is not limited to a case in which the apparatus automatically start fundus image autofocus upon completion of positioning. For example, the apparatus may start fundus image autofocus in accordance with the issuance of an instruction to start focusing by the operator via an operation part such as a switch. When the apparatus starts fundus image autofocus, the fundus image autofocus part 13 executes focusing operation to focus the imaging optical system on an object (the fundus in this embodiment) illuminated by the observation light source 10 as a light source by using the object image obtained by imaging the object using the image sensor 5. During the execution of focusing operation, the apparatus changes imaging settings so as to increase the signal-to-noise ratio of the object image acquired by imaging using the image sensor 5 as compared with that during the non-execution of focusing operation.

In this embodiment, the above operation of changing imaging settings includes increasing the emitted light amount of the observation light source 10 while decreasing the amplification factor of a signal from the image sensor 5 during the execution of focusing operation. First of all, the SN control part 15 changes the emitted light amount of the observation light source 10 from I1 to I2. In this case, the emitted light amount I2 is larger than the emitted light amount I1 (I2>I1). The observation light source 10 emits light at the emitted light amount I2 set by the SN control part 15 (step S104). At the same time with setting the emitted light amount I2, the SN control part 15 changes the amplification factor of the image sensor 5 from S1 to S2. In this case, the amplification factor S2 is smaller than the amplification factor S1 (S1>S2) (step S105).

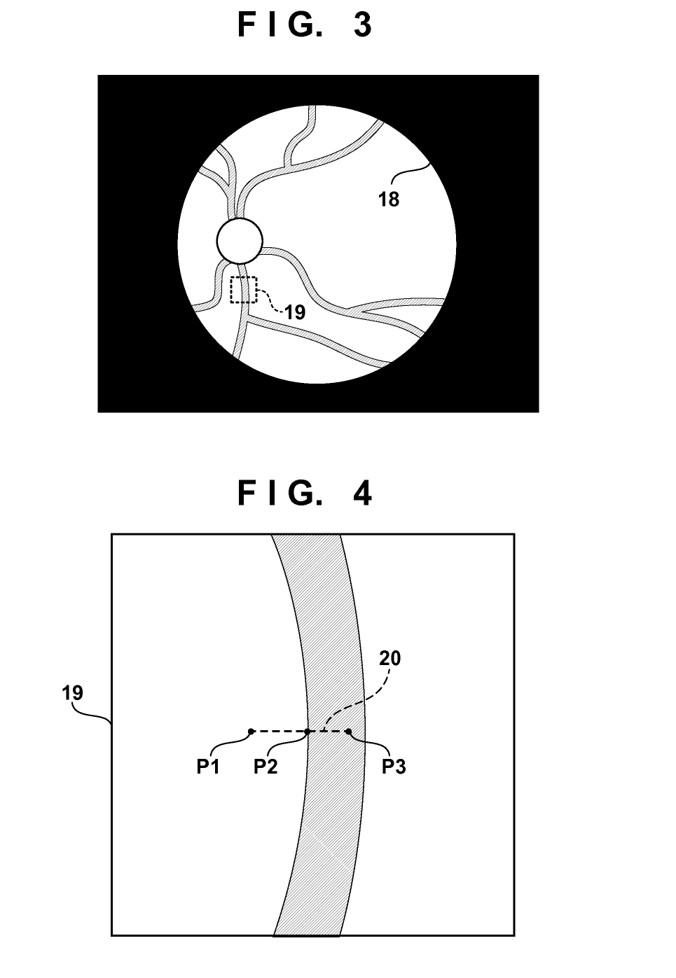

The fundus image autofocus part 13 then executes fundus image autofocus (step S106). In fundus image autofocus, the fundus image autofocus part 13 performs focusing evaluation by using the object image obtained by imaging using the image sensor 5, and automatically focuses the fundus imaging optical system on the fundus Er based on the focusing evaluation. That is, the fundus image autofocus part 13 receives the fundus observation image captured by the image sensor 5, whose setting has been changed in step 5105, while illuminating the fundus Er by using the observation light source 10 whose setting has been changed in step S104. The fundus image autofocus part 13 sets a predetermined area in the received fundus observation image as a focusing evaluation area. In this case, a focusing evaluation area is an area for indicating a specific region of interest in a fundus observation image to which fundus image autofocus is to be executed. FIG. 3 shows an example of a focusing evaluation area in a fundus observation image. Referring to FIG. 3, from a portion where a fundus observation image in a mask 18 is depicted, a portion where medium and large vessels are depicted is set as a focusing evaluation area 19. Note that although the depicted portion of the medium and large vessels is set as the focusing evaluation area 19 in this embodiment, it is not limited to this. For example, another depicted portion such as a papillary region may be set as a focusing evaluation area. In addition, for example, the operator may designate a desired position from a fundus observation image as the focusing evaluation area 19. A predetermined position and region on a fundus observation image may be set as the focusing evaluation area 19.

FIG. 4 shows only the extracted focusing evaluation area 19 set in FIG. 3. The fundus image autofocus part 13 drives the focus lens 3 to search the set focusing evaluation area 19 for a focus lens position at which the maximum focusing evaluation value is obtained. This focusing evaluation value is the magnitude of the tone difference between structures of the fundus observation image depicted in the focusing evaluation area.

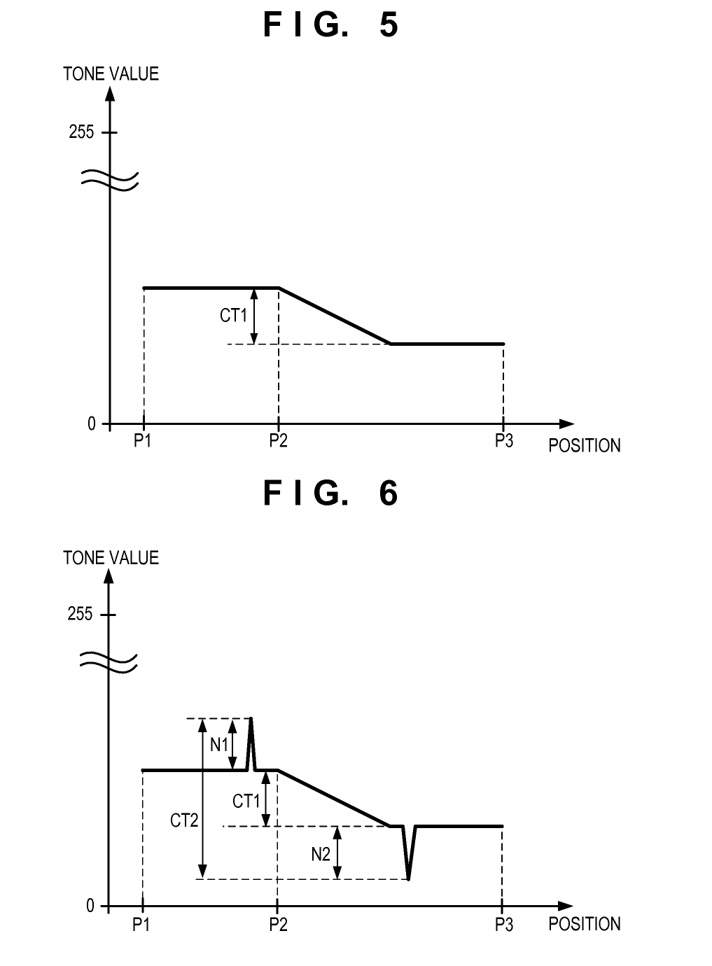

FIG. 5 shows the tone values at positions P1 to P3 on a dotted line 20 shown in FIG. 4. Referring to FIGS. 4 and 5, a portion from the position P1 to the position P2 is a depicted portion of the nerve fiber layer, the position P2 corresponds to a depicted portion of the boundary between the blood vessel and the nerve fiber layer, and a portion from the position P2 to the position P3 is a depicted portion of the blood vessel. Noise of the image sensor 5 is superimposed on each tone value in reality. However, for the sake of descriptive convenience, FIG. 5 shows an ideal state in which each tone value is free from the influence of noise of the image sensor 5.

In this case, a focusing evaluation value is a tone difference CT1 between a nerve fiber layer portion and a blood vessel portion. The fundus image autofocus part 13 searches for a focus lens position at which the focusing evaluation value CT1 is maximized, and moves the focus lens to the position after the search, thereby completing the fundus image autofocus (step S106). Upon completion of the fundus image autofocus, the SN control part 15 resets the emitted light amount of the observation light source 10 from 12 to I1 (step S107). The observation light source 10 then emits light at the emitted light amount I1 changed by the SN control part 15. At the same time, the SN control part 15 resets the amplification factor of the image sensor 5 from S2 to S1 (step S108). The image sensor 5 then captures a fundus observation image with the set amplification factor S1.

An imaging procedure will be described next. Upon completing precise positioning and fundus image autofocus between the eye E and the non-mydriatic fundus camera 100 described above, the operator can perform imaging by operating an imaging start switch (not shown).

When the operator operates the imaging start switch, the fundus camera control part 14 causes the imaging light source 12 to emit light. The imaging illumination light emitted from the imaging light source 12 illuminates the fundus Er upon passing through the fundus illumination optical system extending from the imaging light source 12 to the objective lens 1. The reflected light from the fundus Er illuminated by the imaging light source 12 reaches the image sensor 5 through the fundus imaging optical system extending from the objective lens 1 to the imaging lens 4 through the perforated mirror 2 and the focus lens 3. The display image processing part 16 performs color hue conversion processing and gamma curve calculation processing for the fundus image captured by the image sensor 5, and displays the resultant image on the display part 17.

This embodiment is characterized in that the emitted light amount settings of the observation light source 10 and the amplification factor settings of the image sensor 5 are switched in accordance with whether fundus image autofocus is active in the above manner, and the relationships of the settings are defined as I2>I1 and S1>S2. The following are the reasons why this operation is a feature of this embodiment. Descriptions will be made separately at the times when fundus image autofocus is active and when it is inactive.

### <Fundus Image Autofocus: Inactive>

Observation activity to be performed when fundus image autofocus is inactive is positioning between the eye E and the non-mydriatic fundus camera 100 in the upward, downward, leftward, rightward, forward, and backward directions. The fundus observation image displayed on the display part 17 is required to allow the operator to recognize the relative positional relationship between the non-mydriatic fundus camera 100 and structures of the fundus such as the papilla, macula, and blood vessel when seeing the overall fundus observation image.

For this reason, the emitted light amount of the observation light source 10 is set to a value (I1) as small as possible in the required range described above, while the amplification factor of the image sensor 5 is set to a value (S1) as high as possible. This makes it possible to perform positioning without illuminating an object with an unnecessarily large amount of observation illumination light, that is, without imposing any unnecessarily heavy burden on the object.

On the other hand, setting the amplification factor of the image sensor 5 to the high value (S1) will also amplify noise of the image sensor 5, resulting in a decrease in the signal-to-noise ratio between the fundus observation image and noise of the image sensor 5. This makes the operator recognize the noise of the image sensor 5 when precisely observing part of the fundus observation image upon enlarging it. When performing positioning, however, it is only required to recognize the relative positional relationship between the non-mydriatic fundus camera 100 and structures of the fundus such as the papilla, macula, and blood vessel when seeing the overall fundus observation image. Such a low signal-to-noise ratio poses no serious problem.

### <Fundus Image Autofocus: Active>

In fundus image autofocus, focusing evaluation is performed for a fundus observation image as described in step S106. However, the tone differences between structures of this fundus observation image are very small. For example, the tone difference CT1 between the nerve fiber layer and the blood vessel portion is about 5 to 15. For this reason, noise of the image sensor 5 has a great influence on the focusing accuracy of fundus image autofocus.

The following is how noise influences the focusing accuracy of fundus image autofocus. FIG. 6 shows the tone values at the positions P1 to P3 (FIG. 4) when the emitted light amount I1 of the observation light source 10 and the amplification factor S1 of the image sensor 5 are set in the same manner as when fundus image autofocus is inactive. As is obvious, since the amplification factor S1 of the image sensor 5 is a high value, noise N1 and noise N2 of the image sensor 5 are superimposed on the tone values as compared with that in FIG. 5. The higher the amplification factor of the image sensor 5, the larger the magnitudes of the noise N1 and the noise N2 of the image sensor 5, and vice versa.

When executing fundus image autofocus in such a case, the apparatus calculates a tone difference CT2 caused by the influence of the noise N1 and noise N2 in spite of the necessity to calculate the tone difference CT1, resulting in a great reduction in focusing accuracy. For this reason, at the time of fundus image autofocus operation, the apparatus sets the emitted light amount of the observation light source 10 to a value (I2) as large as possible, and sets the amplification factor of the image sensor 5 to a value (S2) as low as possible. That is, the apparatus makes settings to increase the signal-to-noise ratio between a fundus observation image and noise of the image sensor 5. These settings can implement high focusing accuracy.

The above description is summarized as follows. When fundus image autofocus is inactive, in order to reduce the burden on the object, the apparatus sets the signal-to-noise ratio between a fundus observation image and noise of the image sensor 5 to a low setting (the emitted light amount I1 of the observation light source and the amplification factor S1 of the image sensor 5). When performing positioning in this case, the operator sees the overall fundus observation image to check the relative positional relationship between the structures of the fundus and the apparatus, and hence the low signal-to-noise ratio between the fundus observation image and noise of the image sensor 5 poses no problem. In contrast, when fundus image autofocus is active, the apparatus sets the signal-to-noise ratio between the fundus observation image and noise of the image sensor 5 to a higher ratio than when fundus image autofocus is inactive (that is, the emitted light amount 12 of the observation light source and the amplification factor S2 of the image sensor 5), thereby implementing accurate fundus image autofocus. That is, the relationships between the emitted light amount of the observation light source 10 and the amplification factor of the image sensor 5 when fundus image autofocus is active and those when fundus image autofocus is inactive are defined as I2>I1 and S1>S2.

Note that since the display part 17 displays a fundus observation image even when fundus image autofocus is active, the operator can perform positioning between the eye E and the non-mydriatic fundus camera 100 in the upward, downward, leftward, rightward, forward, and backward directions.

In addition, the above embodiment sets the emitted light amount of the observation light source 10 to 12 and the amplification factor of the image sensor 5 to S2 to increase the signal-to-noise ratio between a fundus observation image and noise of the image sensor 5 when fundus image autofocus is active as compared with when fundus image autofocus is inactive. However, the apparatus may change either the emitted light amount or the amplification factor. For example, in fundus image autofocus, it is possible to increase the signal-to-noise ratio between a fundus observation image and noise of the image sensor 5 by changing the emitted light amount of the observation light source 10 from I1 to I2 while keeping the amplification factor of the image sensor 5 to S1. Likewise, it is possible to increase the signal-to-noise ratio between a fundus observation image and noise of the image sensor 5 by changing the amplification factor of the image sensor 5 from S1 to S2 while keeping the emitted light amount of the observation light source 10 to I1.

The above embodiment changes the amplification factor setting of the image sensor 5 to increase the signal-to-noise ratio between a fundus observation image and noise of the image sensor 5 at the time when fundus image autofocus is active as compared with the time when fundus image autofocus is inactive. However, the present invention is not limited to this. For example, it is also possible to increase the signal-to-noise ratio between a fundus observation image and noise of the image sensor 5 by changing the charge accumulation period of the image sensor 5. In this case, letting SP1 be the charge accumulation period when fundus image autofocus is inactive, and SP2 be the charge accumulation period when fundus image autofocus is active, the apparatus may perform operation so as to satisfy SP1>SP2. Note that the apparatus may change the amplification factor setting and/or the emitted light amount setting in accordance with a change in charge accumulation period.

Although the above embodiment has exemplified the non-mydriatic fundus camera, the present invention is not limited to this. Any type of ophthalmic apparatus which performs fundus image autofocus by using a fundus observation image can perform accurate fundus image autofocus by increasing the signal-to-noise ratio between a fundus observation image and noise of the image sensor 5 when fundus image autofocus is active as compared with when fundus image autofocus is inactive.

Although the above embodiment has exemplified the case of performing fundus image autofocus, it is possible to obtain the same effect as that described above even when the operator manually performs focusing without performing fundus image autofocus while seeing the fundus observation image captured by the image sensor 5. More specifically, when the apparatus shifts to the manual focusing mode in which the apparatus moves the focus lens 3 in accordance with an operation input from the operator to achieve an in-focus state, the imaging control apparatus changes the imaging settings to those for improving the signal-to-noise ratio of the image obtained by imaging. Alternatively, the apparatus may be provided with a detection part which detects that the focus lens 3 is manually operated, and may determine from the detection result obtained by the detection part whether focusing operation is active or inactive. When the focusing part is inactive, the apparatus sets the signal-to-noise ratio between a fundus observation image and noise of the image sensor 5 to a lower setting to reduce the burden on the object. When the focusing part is active, the apparatus sets the signal-to-noise ratio between a fundus observation image and noise of the image sensor 5 to a higher setting than that when focusing operation is inactive. This makes it possible to provide an accurate in-focus state even at the time of manual focusing operation without imposing any burden on the object.

### Second Embodiment

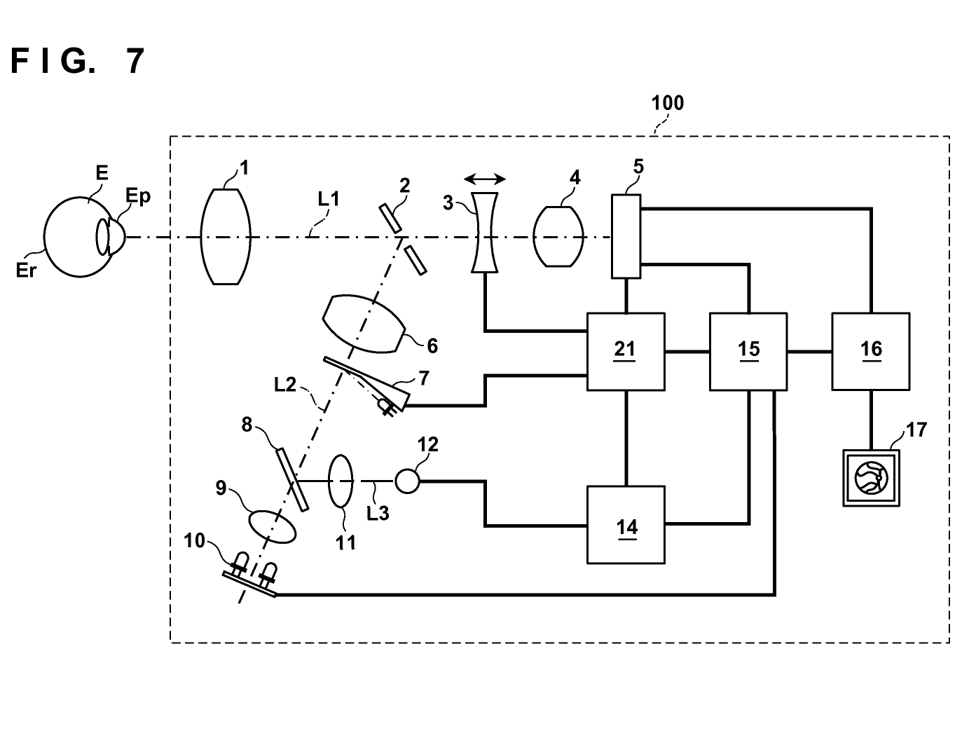

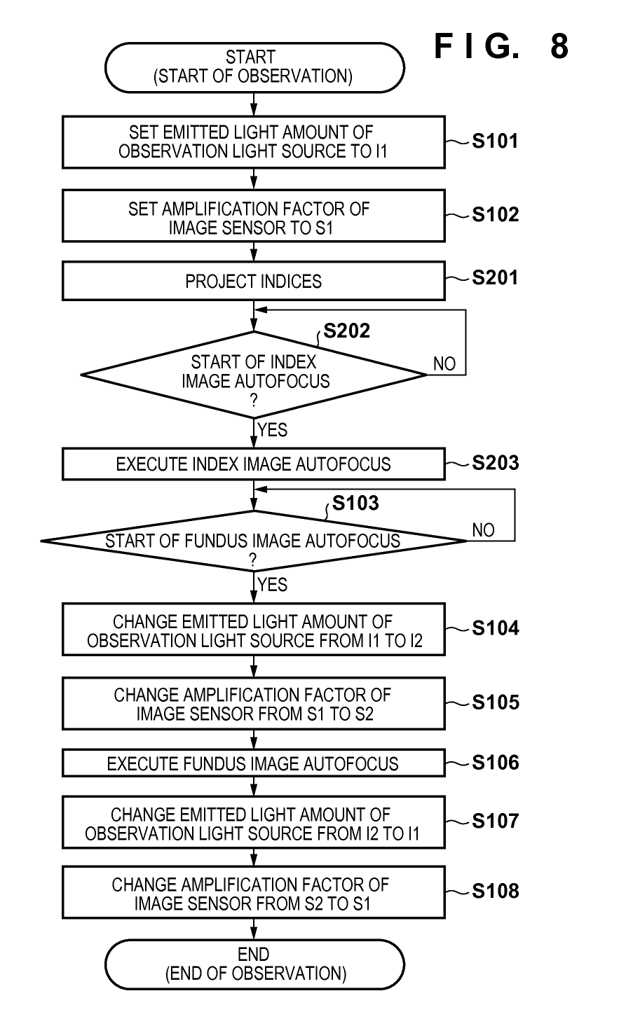

FIG. 7 shows the arrangement of the second embodiment. The same reference numerals as in FIG. 1 denote the same components in FIG. 7. Referring to FIG. 7, an autofocus part 21 replaces the fundus image autofocus part 13 in FIG. 1. The autofocus part 21 is connected to a focus lens 3, an image sensor 5, an index projection part 7, and a fundus camera control part 14, and can perform index image autofocus as the first autofocus and fundus image autofocus as the second autofocus. A non-mydriatic fundus camera 100 performs fundus image autofocus after index image autofocus. However, this camera may selectively execute index image autofocus and fundus image autofocus (that is, the temporal relationship between index image autofocus and fundus image autofocus is arbitrary).

Index image autofocus can accurately perform focusing on the pupil in the splitting direction on the pupil with respect to the ametropia such as the astigmatism of the optical system of the eye to be examined but cannot accurately perform focusing on the indices in directions other than the splitting direction on the pupil. Although index image autofocus has this problem, it can almost specify a focal position in fundus image autofocus to be performed afterward. Performing index image autofocus first and then performing fundus image autofocus can limit a search range at the time of fundus image autofocus. This can greatly shorten the time required for focusing.

Imaging operation is the same as that in the first embodiment, and hence a description of it will be omitted. Operation at the time of observation in the second embodiment will be described with reference to the flowchart of FIG. 8. Note that the flowchart of FIG. 8 indicates the operation of the fundus camera control part 14, an SN control part 15, and the autofocus part 21. The same reference numerals as in FIG. 2 denote the same steps in FIG. 8.

When the operator positions an eye E to be examined in front of an objective lens 1 and starts observation, the SN control part 15 sets the emitted light amount of an observation light source 10 to I1 (step S101) and sets the amplification factor of the image sensor 5 to S1 (step S102). With this operation, the observation light source 10 emits light at the emitted light amount I1, and the image sensor 5 performs imaging with the amplification factor S1. The operator performing positioning between the eye E and the non-mydriatic fundus camera 100 in the upward, downward, leftward, rightward, forward, and backward directions while seeing the fundus observation image displayed on a display part 17.

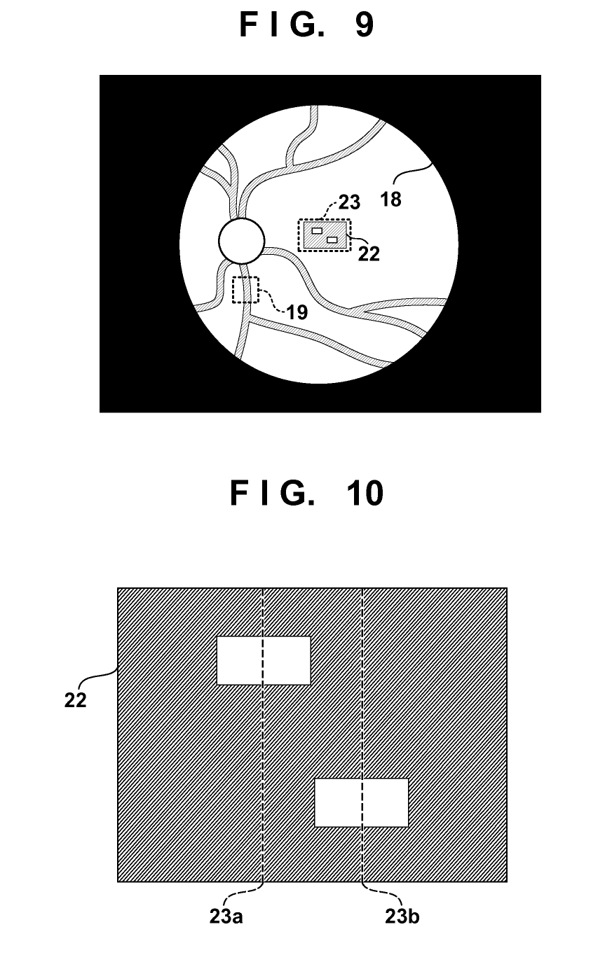

The autofocus part 21 then projects indices from the index projection part 7 onto the eye E (step S201). As shown in FIG. 9, an index image 22 is depicted in the fundus observation image captured by the image sensor 5. When starting index image autofocus (YES in step S202), the autofocus part 21 receives the fundus observation image from the image sensor 5 which is captured with the settings set in steps S101, S102, and S201 (step S203).

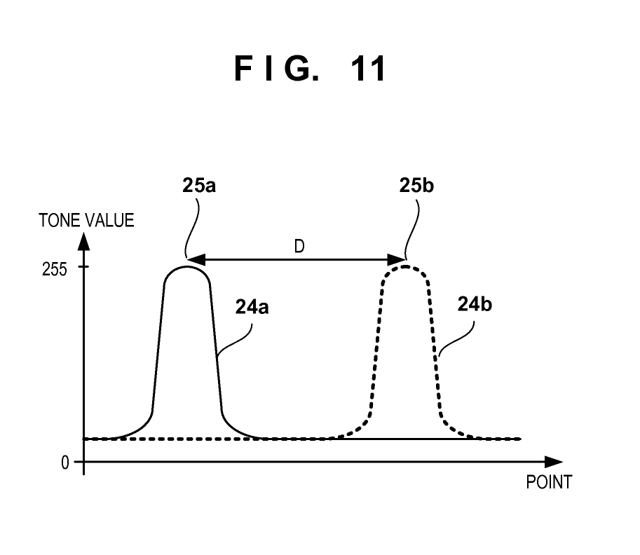

In index image autofocus, first of all, as shown in FIG. 9, the autofocus part 21 sets, as a focusing evaluation area 23, an area with a predetermined size which includes the index image projected on a fundus Er in the fundus observation image. Since the projection position of each index on the fundus is determined in advance in terms of optical design, the focusing evaluation area 23 is fixed, for example, near the center of the fundus observation image. Note that the focusing evaluation area 23 is not limited to the fixing position in the present invention, it is possible to extract an index image from a fundus observation image and set an area with a predetermined size which includes the extracted index image as the focusing evaluation area 23. That is, in the present invention, the position of the focusing evaluation area 23 may be fixed or automatically determined. FIG. 10 shows the extracted focusing evaluation area 23 shown in FIG. 9. FIG. 11 shows tone values on dotted lines 23a and 23b in FIG. 10. Solid line 24a and dotted line 24b respectively represent tone values along the dotted line 23a and tone values along the dotted line 23b. The autofocus part 21 detects a peak position 25a on the solid line 24a and a peak position 25b on the dotted line 24b, and calculates a distance D from the relationship of the two peak positions. The autofocus part 21 then moves the focus lens 3 based on the calculated distance D, thus completing index autofocus.

At the time of index image autofocus, the apparatus does not change the settings of the emitted light amount I1 of the observation light source 10 and the amplification factor S1 of the image sensor 5. This is because, as shown in FIG. 11, since the tone difference on the index image 22 is very large relative to the tone difference between the fundus observation image and the structures, even if the amplification factor S1 of the image sensor 5 is high, the apparatus is robust against the influence of noise of the image sensor 5.

The processing in step S103 including fundus image autofocus and the subsequent steps (steps 103 to S108) is the same as in the first embodiment.

As has been described above, at the time of operation in index image autofocus, in order to reduce the burden on the object, the apparatus makes settings (the emitted light amount I1 of the observation light source and the amplification factor S1 of the image sensor 5) for the low signal-to-noise ratio between a fundus observation image and noise of the image sensor 5. Even if the apparatus makes settings for the low signal-to-noise ratio, since the tone differences on the index image 22 are large from the beginning, the focusing accuracy of index image autofocus does not decrease. In contrast to this, at the time of operation in fundus image autofocus, as described in the first embodiment, the apparatus makes settings (the emitted light amount I2 of the observation light source and the amplification factor S2 of the image sensor 5) for the high signal-to-noise ratio between a fundus observation image and noise of the image sensor 5 to implement accurate fundus image autofocus.

As has been described above, according to the second embodiment, it is possible to reduce the burden on an object by keeping the amount of observation light low during periods other than the execution period of fundus image autofocus. In addition, performing index image autofocus first and then performing fundus image autofocus can greatly shorten the time required for focusing. This can further reduce the burden on the object.

Aspects of the present invention can also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory device (for example, computer-readable storage medium).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-242213, filed Nov. 1, 2012, which is hereby incorporated by reference herein in its entirety.

What is claimed is:

1. An imaging control apparatus comprising:
a focusing unit configured to perform focusing operation to set an imaging optical system in an in-focus state with respect to an object illuminated by a light source by using an object image obtained by imaging the object using an image sensor; and
a change unit configured to change an imaging setting so as to set a signal-to-noise ratio of the object image acquired by the imaging during execution of focusing operation by said focusing unit higher than that during non-execution of the focusing operation.

2. The apparatus according to claim 1, wherein said change unit increases an emitted light amount of the light source during execution of the focusing operation as compared with an emitted light amount during non-execution of the focusing operation.

3. The apparatus according to claim 1, wherein said change unit decreases an amplification factor of a signal from the image sensor during execution of the focusing operation as compared with an amplification factor during non-execution of the focusing operation.

4. The apparatus according to claim 1, wherein said change unit shortens a charge accumulation period of the image sensor during execution of the focusing operation as compared with a charge accumulation period during non-execution of the focusing operation.

5. The apparatus according to claim 1, wherein said focusing unit performs focusing evaluation by using an object image obtained by the imaging and automatically sets the imaging optical system in the in-focus state based on the focusing evaluation.

6. The apparatus according to claim 1, wherein said focusing unit starts the focusing operation in response to a time when a positional relationship between the object and the imaging optical system satisfies a predetermined relationship.

7. The apparatus according to claim 1, wherein said focusing unit executes first autofocus of performing the focusing operation based on an image of an index in an object image which is obtained by projecting the index on the object, and then executes second autofocus of automatically performing the focusing operation based on an image of the object which is obtained from an object image, and
said change unit changes an imaging setting so as to set a signal-to-noise ratio of the object image acquired by the imaging during execution of focusing operation by the second autofocus higher than that during execution of focusing operation by the first autofocus.

8. The apparatus according to claim 7, wherein an imaging setting during focusing operation by the first autofocus is the same as an imaging setting during non-execution of focusing operation by said focusing unit.

9. The apparatus according to claim 1, wherein said focusing unit selectively performs first autofocus of performing the focusing operation based on an image of an index in an object image which is obtained by projecting an index on the object and second autofocus of automatically performing the focusing operation based on an image of the object which is obtained from the object image, and
said change unit changes an imaging setting so as to set a signal-to-noise ratio of the object image acquired by the imaging during execution of focusing operation by the second autofocus higher than that during execution of focusing operation by the first autofocus.

10. An ophthalmic apparatus comprising:
the imaging control apparatus defined in claim 1;
the light source;
the image sensor; and
the imaging optical system,
wherein a fundus image is captured as the object image.

11. An imaging control method comprising:
a step of causing focusing means to perform focusing operation to set an imaging optical system in an in-focus state with respect to an object illuminated by a light source by using an object image obtained by imaging the object using an image sensor; and
a step of causing change means to change an imaging setting so as to set a signal-to-noise ratio of the object image acquired by the imaging during execution of the focusing operation higher than that during non-execution of the focusing operation.

12. A non-transitory computer-readable storage medium storing a program for causing a computer to execute each step in an imaging control method defined in claim 11.
